(19) United States
(12) Patent Application Publication
(10) Pub. No.: US 2022/0277121 A1
(43) Pub. Date: Sep. 1, 2022

(54) AUTOMATIC GENERATION OF DISTILLATION MODELS FOR NON-IDEAL BEHAVIOR IN BLENDED FUELS

(71) Applicant: Bryan Research & Engineering, LLC, Bryan, TX (US)

(72) Inventors: Barry Burr, Bryan, TX (US); Justin Slagle, College Station, TX (US); Adam Spencer, Arlington, TX (US)

(73) Assignee: Bryan Research & Engineering, LLC, Bryan, TX (US)

(21) Appl. No.: 17/188,893

(22) Filed: Mar. 1, 2021

(51) Int. Cl. G06F 30/28

(52) U.S. Cl. CPC G06F 30/28; G06F 2113/08

(57) ABSTRACT

Apparatus and associated methods relate to generating, from a predetermined distillation profile (DP_{BASE-P}) of a multicomponent base fluid, a thermodynamically accurate distillation profile (DP_{MIX}) of a mixture of the base fluid and at least one additive. In an illustrative example, at least one calibrating parameter (P) of a distillation model (DM) is determined according to DP_{BASE-P}. The base fluid and the mixture may, for example, be represented by a base composition profile (CP_{BASE}) and mixture composition profile (CP_{MIX}), respectively, of pure chemical components. The DM may, for example, be calibrated to DP_{BASE-P} to generate a distillation profile of the base fluid as a function of CP_{BASE} and P. The calibrated DM may, for example, be configured to generate DP_{MIX} as a function of P and CP_{MIX}. Various embodiments may advantageously enable rapid characterization of a mixture from a known distillation profile of the base fluid.

### CROSS-REFERENCE TO RELATED APPLICATIONS

This application contains subject matter related to the following:
U.S. patent application Ser. No. 15/904,334, titled “Characterization of Complex Hydrocarbon Mixtures,” filed by Martinis, et al., on Feb. 24, 2018, and issued as U.S. Pat. No. 10,566,079;
U.S. patent application Ser. No. 14/287,980, titled “Characterization of Complex Hydrocarbon Mixtures for Process Simulation,” filed by Martinis, et al., on May 27, 2014;
U.S. Patent Application No. 61/886,756, titled “Characterization of Complex Hydrocarbon Mixtures for Process Simulation,” filed by Martinis, et al., on Oct. 4, 2013;
U.S. patent application Ser. No. 16/692,085, titled “Composition Tracking of Mixed Species in Chemical Processes,” filed by Hull, et al., on Nov. 22, 2019; and,
U.S. Patent Application Ser. No. 62/904,806, titled “Composition Tracking of Mixed Species in Chemical Processes,” filed by Hull, et al., on Sep. 24, 2019.

This application incorporates the entire contents of the foregoing application(s) herein by reference.

### TECHNICAL FIELD

Various embodiments relate generally to characterization of distillation properties for multicomponent fluid mixtures.

### BACKGROUND

Distillation processes may be used to separate components or substances from a liquid mixture by combination of (e.g., successive) boiling and condensation. Distillation experiments may, for example, take advantage of variations and relative volatility of components of a mixture to identify attributes of one or more components, the mixture as a whole, or some combination thereof. Results of a distillation experiment may, for example, be represented as a plot comparing quantity and energy.

Petroleum products may, for example, be characterized using a batch distillation process. For example, the American Society for Testing and Material (ASTM) defines a standard distillation test referred to as ASTM D86. The ASTM D86 distillation test may be used to estimate volatility characteristics of hydrocarbon-based mixtures. The results of the distillation test may, for example, give information on a mixture's composition, properties, and/or behavior during storage and/or use (e.g., as a combustible fuel).

### SUMMARY

Apparatus and associated methods relate to generating, from a predetermined distillation profile (DP_{BASE-P}) of a multicomponent base fluid, a thermodynamically accurate distillation profile (DP_{MIX}) of a mixture of the base fluid and at least one additive. In an illustrative example, at least one calibrating parameter (P) of a distillation model (DM) is determined according to DP_{BASE-P}. The base fluid and the mixture may, for example, be represented by a base composition profile (CP_{BASE}) and mixture composition profile (CP_{MIX}), respectively, of pure chemical components. The DM may, for example, be calibrated to DP_{BASE-P} to generate a distillation profile of the base fluid as a function of CP_{BASE} and P. The calibrated DM may, for example, be configured to generate DP_{MIX} as a function of P and CP_{MIX}. Various embodiments may advantageously enable rapid characterization of a mixture from a known distillation profile of the base fluid.

Various embodiments may achieve one or more advantages. For example, some embodiments may advantageously generate a distillation model that is specifically tuned to unique characteristics of a base fluid based on a predetermined distillation profile of the base fluid. Various embodiments may advantageously produce a distillation profile of the mixture of the base fluid with at least one additive. Various embodiments may advantageously enable a (general purpose) computer system to accurately perform a distillation experiment on a fluid mixture that would otherwise require costly and time-consuming physical experiments. Various embodiments may advantageously enable virtual distillation property characterization laboratory experiments to be performed using one or more methods that are more efficient and/or more cost effective, for example, than methods and apparatus used in manual, physical distillation experiments. Various embodiments may advantageously enable virtual distillation property characterization simulations to be performed using one or more methods that are more thermodynamically robust (e.g., producing more accurate results) than current simulation methods, at least in relation to non-ideal behavior of blended fluids (e.g., fuels).

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary system for automatic generation of distillation models for non-ideal behavior in blended fluids employed in an illustrative use-case scenario.

FIG. 2 depicts an exemplary method for automatically generating a distillation profile of a blended multicomponent fluid based on a predetermined distillation profile of a base fluid.

FIG. 3 depicts an exemplary method of generating a composition profile for the multicomponent base fluid based on a predetermined distillation model of the base fluid.

FIG. 4 depicts an exemplary method of generating a calibrated distillation model based on the composition profile of pure components for the base fluid and a predetermined distillation profile of the base fluid.

FIG. 5 depicts an exemplary method of generating an ASTM D86 distillation profile for a base fuel blended with an additive based on a predetermined ASTM D86 distillation profile of the base fuel.

FIG. 6 depicts an exemplary method of determining a reflux ratio profile (RRP) as at least one calibrating parameter for a distillation model based on a predetermined distillation profile and a composition profile of a base fluid.

FIG. 7 depicts an exemplary method of determining a heat transfer coefficient (K) as at least one calibrating parameter for a distillation model based on a predetermined distillation profile and a composition profile of a base fluid.

FIG. 8 depicts an exemplary hardware block diagram of an automatic laboratory module 110.

Like reference symbols in the various drawings indicate like elements.

### DETAILED DESCRIPTION OF ILLUSTRATIVE EMBODIMENTS

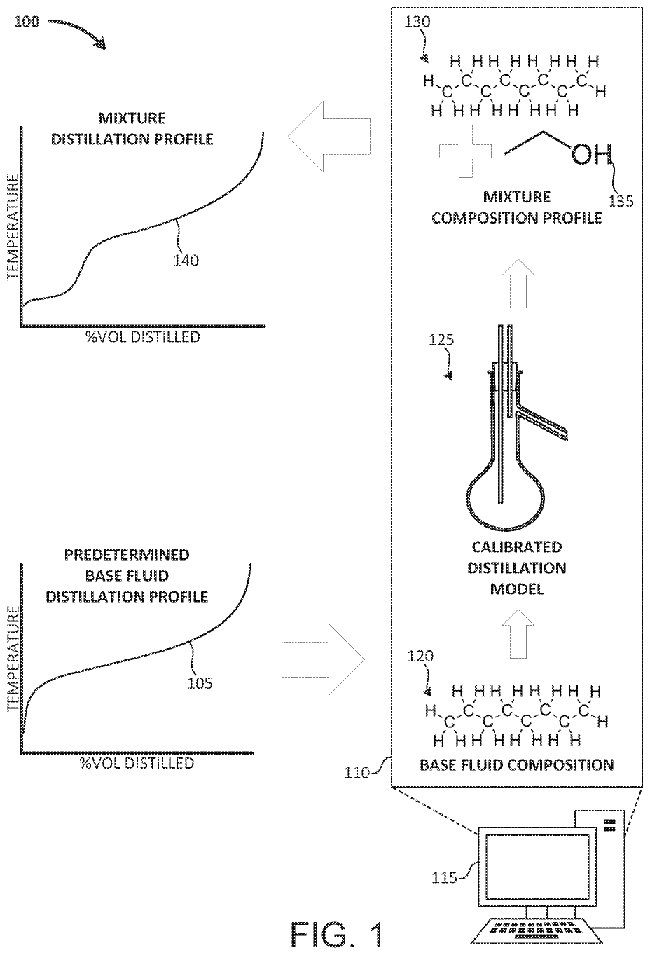

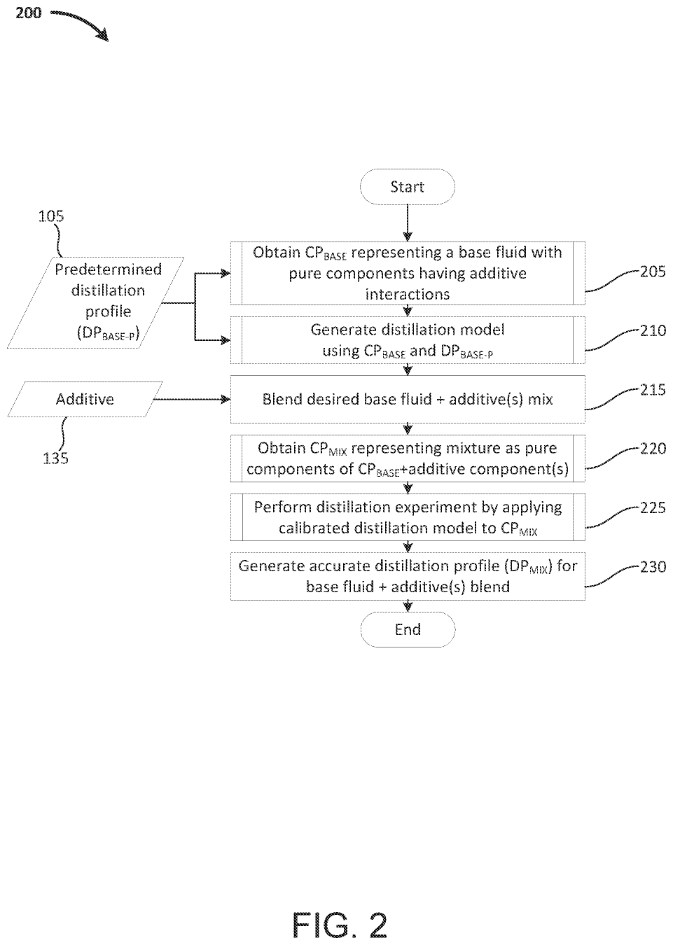

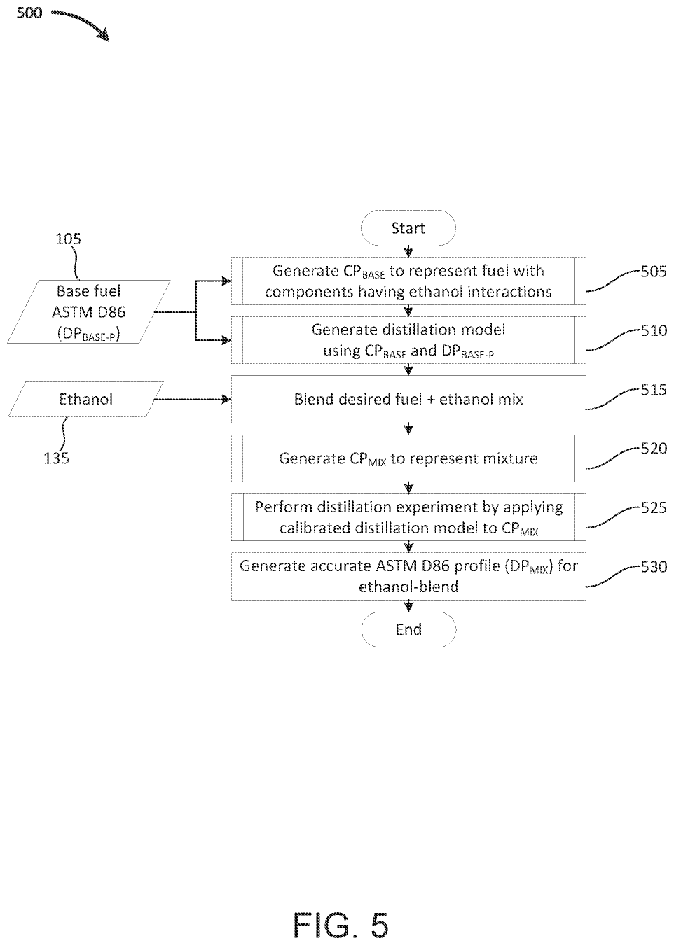

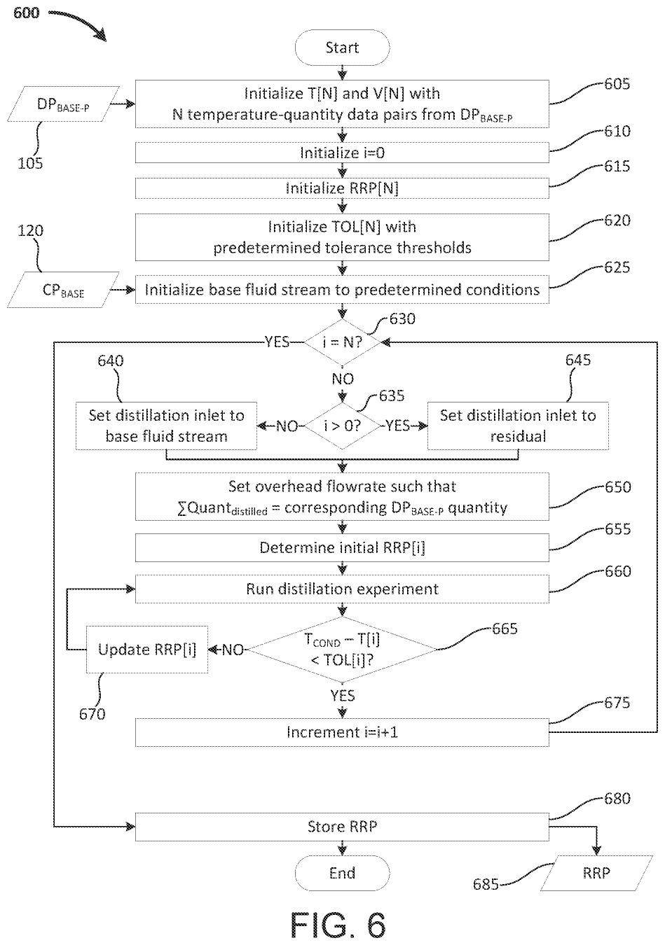

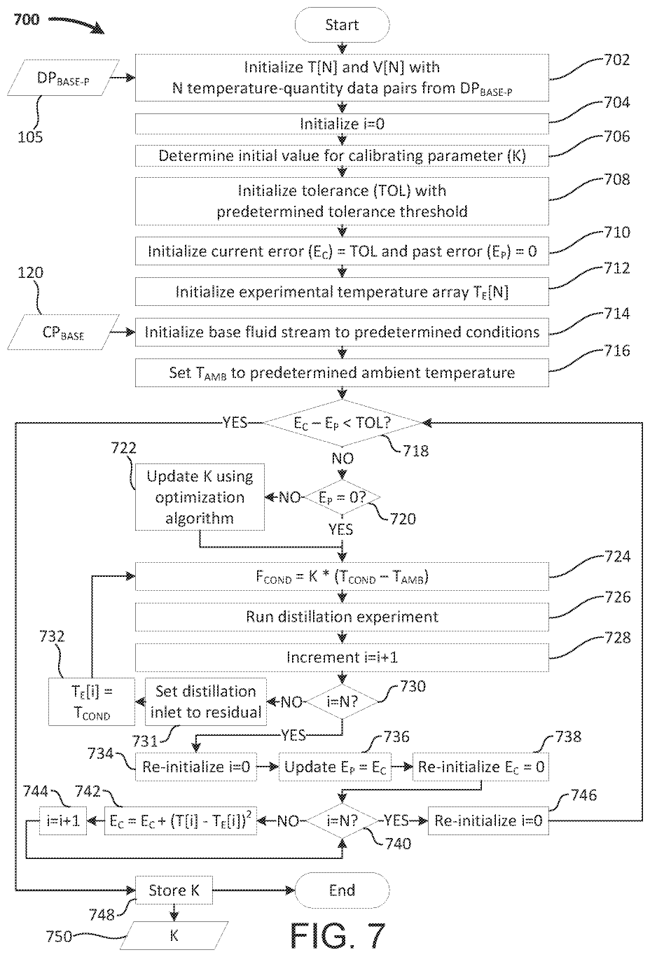

To aid understanding, this document is organized as follows. First, to help introduce discussion of various embodiments, an automatic distillation model generation system is introduced with reference to FIG. 1. Second, that introduction leads into a description with reference to FIGS. 2-4 of exemplary methods which may be used in automatically generating a distillation profile of a base fluid blended with an additive(s) from a predetermined distillation profile of the base fluid. Third, with reference to FIG. 5, application to determination of an ASTM D86 distillation profile of a fuel blend based on an ASTM D86 distillation profile of a base fuel is discussed. Fourth, with reference to FIGS. 6-7, the discussion turns to exemplary methods of determining at least one calibrating parameter of an automatically generated distillation model in various embodiments. Finally, the document discusses further embodiments, exemplary applications and aspects relating to automatic generation of distillation models for non-ideal behavior of blended fluids.

FIG. 1 depicts an exemplary system for automatic generation of distillation models for non-ideal behavior in blended fluids employed in an illustrative use-case scenario. In the depicted system 100, a predetermined distillation profile 105 of a base fluid is provided to an automatic laboratory module 110 implemented in an exemplary computer 115. The automatic laboratory module 110 determines, from the predetermined distillation profile 105, a base fluid composition profile 120 of the base fluid. From the composition profile 120 and the predetermined distillation profile 105, the automatic laboratory module 110 generates a calibrated distillation model 125. A mixture composition profile 130 is determined which includes the base fluid composition profile 120 and components representing one or more additives 135. From the calibrated distillation model 125 and the composition profile 130, the model is applied to generate a distillation profile 140 for the mixture 130.

In the depicted example, the composition profile 120 of the base fluid includes octane. The base fluid may, by way of example and not limitation, be a hydrocarbon fluid such as a petroleum-based fuel. In the depicted example, the additive 135 includes ethanol. As depicted, the predetermined distillation profile 105 and the mixture distillation profile 140 are distillation profiles corresponding to the ASTM D86 fuel distillation standard. The distillation profiles may, for example, correspond quantity (e.g., percent volume distilled as shown, mole fraction, mass fraction) to temperature. The distillation model 125 may, for example, represent an experimental distillation process and apparatus. For example, the distillation model 125 may correspond to the ASTM D86 standard fuel distillation physical apparatus and process.

In various embodiments the automatic laboratory module 110 may advantageously generate a distillation model 125 that is specifically tuned to unique characteristics of a base fluid based on the predetermined distillation profile 105 of the base fluid. Various embodiments may advantageously produce a distillation profile 140 of the mixture of the base fluid with at least one additive 135. Accordingly, various embodiments may advantageously enable a (general purpose) computer system 115 to accurately perform a distillation experiment on a fluid mixture that would otherwise have required costly and time-consuming physical experiments. The automatic laboratory module 110 may advantageously enable virtual distillation property characterization laboratory experiments to be performed using one or more methods that are more efficient and/or more cost effective, for example, than methods and apparatus used in manual, physical distillation experiments.

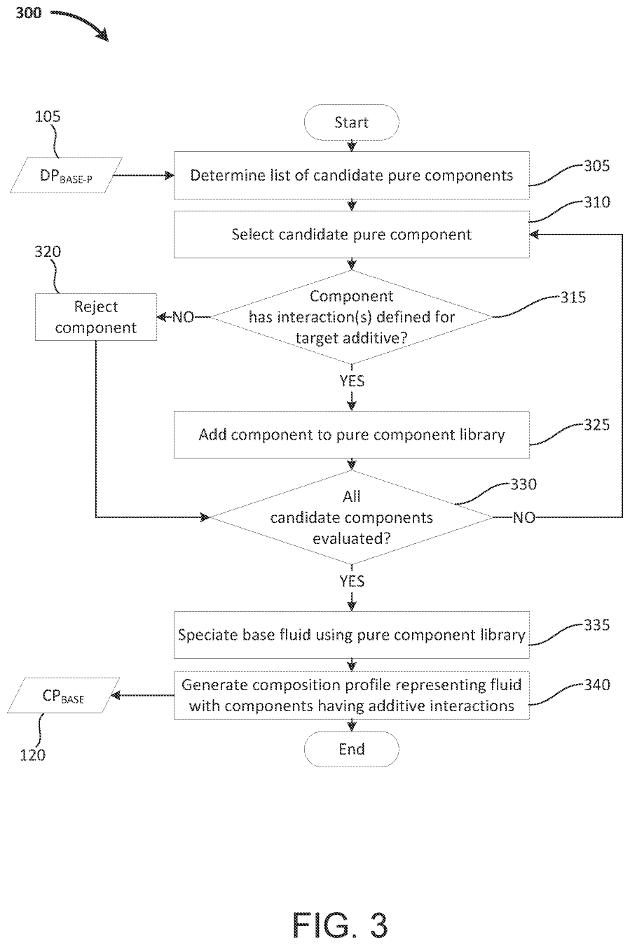

FIG. 2 depicts an exemplary method for automatically generating a distillation profile of a blended multicomponent fluid based on a predetermined distillation profile of a base fluid. The method 200 begins by obtaining 205 a base composition profile (CP_{BASE}) 120 to represent the base fluid using the predetermined distillation profile (DP_{BASE-P}) 105. In various embodiments the base composition profile 105 may, by way of example and not limitation, be obtained by generation of a composition profile using method 300 as described in FIG. 3, by a physical speciation analysis, retrieval of a predetermined speciation profile, or some combination thereof. In various embodiments the composition profile may, for example, define and/or reference predefined molecular structures, one or more corresponding chemical properties (e.g., boiling point, flash point, molecular weight) of each chemical species, and/or corresponding (relative) quantity (e.g., volume, concentration) of each chemical species.

As depicted, the first composition profile 120 is generated using pure components having predefined interactions with the additive(s) 135. In various embodiments the predefined interactions may, by way of example and not limitation, include binary interaction parameters and/or activity coefficients, or other appropriate chemical interaction relationship(s). In various embodiments interaction parameters/coefficients may relate to (e.g., be calculated and/or determined according to) one or more desired equation of state (EOS) related coefficient models such as, by way of example and not limitation, the volume-translated Peng-Robinson (VTPR) estimation method, the Wilson activity coefficient model, the non-random two-liquid theory (NRTL), the universal quasichemical activity coefficient model (UNIQUAC), the Margules Gibbs free energy model, or some combination thereof. Parameters may, for example, be UNIQUAC functional-group activity coefficients (UNIFAC) and/or infinite-dilution activity coefficients.

Pure components (which may also be referred to as ‘real’ components) may, for example, represent molecules existing in the real world (e.g., benzene, octane, water, oxygen). Various embodiments may, by way of example and not limitation, specifically represent some or all composition profiles by way of pure (real) components and not by pseudocomponents. Pseudocomponents may, for example, represent grouped constituents of a mixture as a single, non-existent (non-real) component with chemical properties representative of the grouped constituents (e.g., to reduce complexity of a mixture with many distinct constituent molecules). Pseudocomponents may introduce errors when dealing, for example, with mixtures having constituents with polar or polar-like behavior, with mixtures for which the pseudocomponents were not intended, or some combination thereof. Accordingly, various embodiments may advantageously enable generation and application of distillation model(s) with increased accuracy by use of pure (real) components in composition profiles.

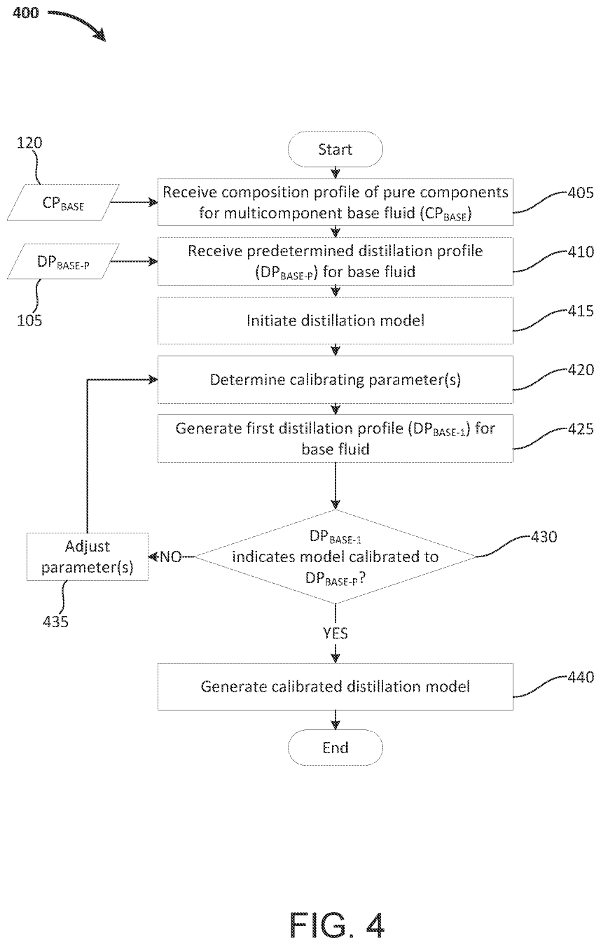

A distillation model 125 is generated 210 using the predetermined distillation profile 105 and the base composition profile 120. In various embodiments the distillation model 125 may be generated, by way of example and not limitation, using the method 400 described in relation to FIG. 4, the method 600 described in relation to FIG. 6, and/or the method 700 described in relation to FIG. 7. A mixture is then created by virtually blending 215 the base fluid and additive(s) 135 in desired proportions. A mixture composition profile 130 (CP_{MIX}) is then obtained 220 representing the mixture as a composition of pure components. The mixture composition profile 130 may, by way of example and not limitation, be generated as discussed in relation to step 210, retrieved from a datastore, determined by manual and/or automatic addition of pure component(s) of the additive to the base composition profile 120, or some combination thereof.

The distillation experiment represented by the distillation model 125 is then performed 225 (e.g., by automatic laboratory module 110) by applying the distillation model 125 to the mixture composition profile 130. From the results of the distillation experiment, a mixture distillation profile (DP_{MIX}) 140 is generated 230. The mixture distillation profile 140 may, for example, represent distillation characteristics of the mixture of the base fluid and additive(s) 135 within a desired range of accuracy. In various embodiments, the use of pure components may advantageously permit increased accuracy in the virtual distillation process as compared to a corresponding physical distillation process. In various embodiments, the combination of composition profiles using pure components for which interaction parameters with the additive component(s) are defined with individual calibration of the distillation model 125 to the predetermined distillation profile 120 (e.g., derived from a physical experiment) of the base fluid may advantageously enable a computing apparatus (e.g., general purpose computer 115) to quickly and efficiently conduct a virtual distillation experiment with a level of accuracy sufficient for use in designing a large-scale industrial chemical process(es) and/or facility/facilities.

FIG. 3 depicts an exemplary method of generating a composition profile for the multicomponent base fluid based on a predetermined distillation model of the base fluid. In the depicted method 300, a list of candidate pure components 305 are determined suitable for a predetermined distillation profile (DP_{BASE-P}) 105. In various embodiments the list of candidate pure components may, for example, be retrieved from a datastore (e.g., previously automatically and/or manually selected and stored). The candidate pure components may, for example, be predefined components representing potential molecules (potentially) present and/or of interest in a mixture (e.g., a base fluid, additive, or some combination thereof). The candidate pure components may, for example, be indirectly included by inclusion of one or more mixtures containing predefined quantities (e.g., fractions) of pure components, such as is described, for example, in U.S. patent application Ser. No. 16/692,085, titled “Composition Tracking of Mixed Species in Chemical Processes,” filed by Hull, et al., on Nov. 22, 2019 and/or U.S. Patent Application Ser. No. 62/904,806, titled “Composition Tracking of Mixed Species in Chemical Processes,” filed by Hull, et al., on Sep. 24, 2019, the entire contents of which are incorporated herein by reference.

A candidate pure component is selected 310. In the depicted example, the selected candidate pure component 310 is then evaluated for the presence of at least one predefined interaction parameter (e.g., binary interaction parameter, activity coefficient) between the selected pure component and at least one (pure) component of at least one additive 135 which will be mixed with the base fluid. If the component has no predefined interaction(s), or has no interaction(s) of interest, then the component is rejected 320. If the component does have at least one predefined interaction (of interest), then the component is added 325 to a pure component library for (potential) use in generating a composition profile.

When a candidate component is rejected 320 or added 325 to the pure component library, if all candidate components have not yet been evaluated 330, then another candidate pure component is selected 310. Once all candidate components have been evaluated 330, then the base fluid is speciated using the pure component library built in steps 305-330. The speciation step may, for example, determine relative quantities (e.g., mole, mass, and/or volume fractions, ratios, and/or percentages) of at least some of the components from the pure component library adequate to represent the base fluid. In exemplary embodiments, the speciation step 335 may, by way of example and not limitation, be performed using methods such as are described in U.S. patent application Ser. No. 15/904,334, titled “Characterization of Complex Hydrocarbon Mixtures,” filed by Martinis, et al., on Feb. 24, 2018, and issued as U.S. Pat. No. 10,566,079; U.S. patent application Ser. No. 14/287,980, titled “Characterization of Complex Hydrocarbon Mixtures for Process Simulation,” filed by Martinis, et al., on May 27, 2014; and/or U.S. Patent Application No. 61/886,756, titled “Characterization of Complex Hydrocarbon Mixtures for Process Simulation,” filed by Martinis, et al., on Oct. 4, 2013; the entire contents of which applications are incorporated herein by reference. In various embodiments the speciation step 335 may, by way of example and not limitation, accept a distillation profile (e.g., DP_{BASE-P} 105) of the base fluid, or information determined therefrom, and density (e.g., American Petroleum Institute (API) gravity) of the base fluid as inputs.

The composition profile 120 is then generated 340, representing the base fluid using pure components having predefined interactions with the additive(s) of interest. Accordingly, various embodiments may advantageously provide composition profiles which accurately represent a multicomponent fluid of interest in, for example, various thermodynamic equations of state. Various such embodiments may, therefore, advantageously enable thermodynamically virtual distillation experiments to be performed. In various embodiments the method 300 may, by way of example and not limitation, be applied to one or more additives, a mixture of the base fluid and one or more additives, or some combination thereof.

FIG. 4 depicts an exemplary method of generating a calibrated distillation model based on the composition profile of pure components for the base fluid and a predetermined distillation profile of the base fluid. In the depicted method 400, the base composition profile (CP_{BASE}) 120 of pure components represents the multicomponent base fluid is received 405. In various embodiments the base composition profile 120 may, by way of example and not limitation, be received as an output of method 300 described in relation to FIG. 3. The predetermined distillation profile (DP_{BASE-P}) 105 is received 410 for the base fluid. The predetermined distillation profile 105 may, for example, be experimentally derived in a physical laboratory (e.g., according to the ASTM D86 standard distillation experiment). Once the base composition profile 120 and the predetermined distillation profile 105 are received, a distillation model 125 is initiated 415 (e.g., by automatic laboratory module 110).

At least one calibrating parameter of the distillation model 125 is determined 420. In various embodiments the calibrating parameter may, by way of example and not limitation, be initiated, calculated, ‘guessed’ according to predetermined rules, input, retrieved from a lookup table, or some combination thereof. In various embodiments the calibrating parameter(s) may include, by way of example and not limitation, a reflux ratio (profile) (e.g., as discussed in relation to FIG. 6), a heat transfer coefficient (e.g., as discussed in relation to FIG. 7), or some combination thereof.

A virtual distillation experiment is performed to generate 425 a test distillation profile (DP_{BASE-1}) for the base fluid using the distillation model 125. The test distillation profile is then compared to the predetermined distillation profile 105 to determine if the distillation model 125 is calibrated 430 to the predetermined distillation profile 105 and, thereby, to the base fluid. In various embodiments the predetermined distillation profile 105 and the test distillation profile may, for example, be compared according to one or more predetermined tolerance thresholds (e.g., for pointwise error, summed (squared) error).

If the distillation model 125 is not calibrated 430 to the predetermined distillation profile 105, then the calibrating parameter(s) is adjusted 435. In various embodiments the calibrating parameter(s) may, by way of example and not limitation, be adjusted incrementally, according to an optimization algorithm (e.g., to minimize calculated error between the test distillation profile and the predetermined distillation profile 105), or some combination thereof. Once the distillation model 125 is calibrated 430 to the predetermined distillation profile 105, the calibrated distillation model is generated 440. For example, the distillation model 125 and/or the calibrating parameter(s) may be stored to one or more data stores for application to one or more mixtures with the base composition profile 120. In various embodiments the method 300 may advantageously enable generation of a virtual distillation model 125 which is calibrated to accurately represent distillation characteristics of a mixture of additive(s) 135 with a specific base fluid.

In various embodiments the distillation model may, by way of example and not limitation, include a batch distillation model, a dynamic distillation model, or some combination thereof. In various embodiments the distillation model may, for example, represent a distillation column, a distillation flask, or some combination thereof. The distillation model may, for example, correspond to (selected) thermodynamic properties of a physical ASTM D86 experimental apparatus.

In various embodiments the distillation model may, for example, include operations representing chemical process steps occurring to incoming streams represented by a composition profile (e.g., base composition profile 120). In some embodiments the distillation model may, for example, incrementally vary temperature in a distillation process and determine corresponding chemical properties (e.g., quantity), and/or incrementally vary time in a distillation process and determine corresponding chemical properties (e.g., temperature, quantity). In various embodiments the distillation model may, by way of example and not limitation, include steady-state process simulation, dynamic process simulation, or some combination thereof.

FIG. 5 depicts an exemplary method of generating an ASTM D86 distillation profile for a base fuel blended with an additive based on a predetermined ASTM D86 distillation profile of the base fuel. The method 500 may, for example, be an adaptation of method 200 to generation of an ASTM D86 distillation profile for a base fuel from the predetermined ASTM D86 distillation profile of the base fuel. The method 500 begins by generating 505 the base composition profile (CP_{BASE}) 120 of pure components for a multicomponent base fuel (e.g., gasoline, diesel, jet fuel) from the predetermined distillation profile 105 of the base fuel. As depicted, the predetermined distillation profile 105 is (derived from) an ASTM D86 distillation profile of the base fuel. In various embodiments the base composition profile 120 may, for example, be generated according to method 300 described in relation to FIG. 3 (e.g., including using oil speciation methods described in previously incorporated related U.S. patent applications).

The predetermined base composition profile 105 and base composition profile 120 are used to generate 510 a distillation model 125. The distillation model 125 may, for example, correspond to a distillation experimental apparatus (e.g., an ASTM D86 flask and distillation column). The distillation model 125 may be generated, for example, by determining one or more calibration parameters such that the distillation model 125 produces a test distillation model of the base fluid from the base composition profile 120 that corresponds to the predetermined distillation profile 105 within at least one tolerance threshold.

An additive 135 is then blended 515 with the fuel to create a desired mixture. In the depicted example, the additive 135 is ethanol. The resulting blended fuel may, for example, be an ethanol fuel (e.g., 5%, 10%, or more ethanol gasoline). In various embodiments the additive 135 may, for example, be propanol, methanol, MTBE, or some combination thereof. A mixture composition profile 130 is generated 520 to represent the blended fuel mixture. For example, the mixture composition profile 130 may include the pure components of the base composition profile 120 with equivalent ratios therebetween, combined with the pure component(s) representing the additive. In the depicted example, the mixture composition profile 130 may, by way of example and not limitation, be the base composition profile 120 (e.g., 95% of the blended mixture distributed among the components of the base composition according to the base composition profile 120) plus at least one ethanol component in relative proportion to the blended mixture (e.g., 5% ethanol).

A distillation experiment is then performed 525 by applying the calibrated distillation model 125 to the mixture composition profile 130. The distillation experiment may, for example, be an electronic distillation experiment achieving results corresponding to a physical distillation experiment but implemented using cost-effective and/or time efficient methods, including those disclosed herein. From the results of the distillation experiment 525, a mixture distillation profile (DP_{MIX}) 140 is generated 530 for the blended fuel (e.g., ethanol blend, as depicted). As depicted, the mixture distillation profile 140 is an electronically generated ASTM D86 distillation curve. Accordingly, various embodiments may advantageously enable accurate ASTM D86 characterization of a (proposed) fuel blend without costly and time-consuming characterization.

Use of pure component composition profiles and a distillation model 125 calibrated to the predetermined distillation profile 105 may advantageously enable a (general purpose) computer system to accurately determine the mixture distillation profile. For example, an individual distillation profile may have unique features dependent, by way of example and not limitation, on the laboratory setup, ambient conditions at the time the experiment was performed, unique features of the fluid characterized (including features, for example, which may not be appreciated in one or more standard analyses), or some combination thereof. The distillation profile may, thus, even while conforming to a known standard (e.g., ASTM D86), represent have specific features that may reduce the value (e.g., for analysis of the effect of blending a base fluid with one or more additives) of distillation profiles generated by generic distillation models and/or experimental setups which are not specifically calibrated to replicate the experimental conditions (e.g., apparatus, ambient conditions, base fuel) which produced the original distillation profile of the base fluid. Accordingly, various embodiments may advantageously enable a user to quickly, economically, and effectively generate a distillation model capable of performing an electronic distillation experiment that is calibrated to the conditions that produced the original predetermined distillation model 105. Various embodiments may, thus, advantageously enable rapid and cost-effective analysis of multiple proposed blends with accuracy, for example, at least suitable for making process design decisions. In some embodiments rapid and cost-effective analysis may advantageously allow greater optimization and/or customization of fluid blends (e.g., fuel blends) for specific use cases, needs, processes, and/or environments.

FIG. 6 depicts an exemplary method of determining a reflux ratio profile (RRP) as at least one calibrating parameter for a distillation model based on a predetermined distillation profile and a composition profile of a base fluid. The method 600 begins by initializing 605 corresponding arrays of temperature (T[N]) and quantity (V[N], e.g., volume fraction, mole fraction, mass fraction) from the base distillation profile (DP_{BASE-P}) 105, where N is the length of the arrays corresponding to the number of temperature-quantity data pairs used to represent the base distillation profile 105. A counter variable (i) is initialized 610 (e.g., to zero, as depicted). A reflux ratio profile (RRP[N]) of length N is initialized 615. A tolerance array (TOL[N]) of length N is initialized 620 with predetermined tolerance thresholds. The tolerance thresholds may, for example, be input manually, be calculated according to predetermined rules, be retrieved from one or more data stores, or some combination thereof.

A base fluid stream corresponding to the base composition profile 120 for the process simulation is initialized 625 to predetermined conditions (e.g., temperature, relative quantities of components, vapor fractions). The base composition profile 120 may, for example, be determined by method 300 as described in relation to FIG. 3, including, for example, using speciation method(s) described in relation thereto. The counter variable (i) is compared 630 to the number of temperature-quantity data pairs.

If the last data pair has not been reached (corresponding to i=N), then the counter variable is checked 635 to determine if a current pass corresponds to an initial pass through an RRP determination loop (steps 630-675). If the counter variable indicates that the current pass is the first pass through the loop, then parameters of the inlet of the distillation model 125 are set to match the base fluid stream initialized in step 625. If the counter variable indicates that the current pass is a subsequent pass through the loop, then the inlet of the distillation model 125 is set to parameters corresponding to residuals left after the previous pass through the loop for the distillation model 125.

An overhead flow rate for the distillation model 125 is set 650 such that the sum of the quantities distilled (ΣQuant_{distilled}) is equal to a corresponding quantity (e.g., corresponding by temperature point) in the predetermined distillation profile 105. A corresponding reflux ratio (RRP[i]) is determined 655. The reflux ratio may be determined, for example, by an initial guess algorithm, a standard initial value (e.g., zero, some other value, a value retrieved from a look up table), a manually input value, or some combination thereof The distillation experiment represented by the distillation model 125 is then performed 660 using the determined reflux ratio. A difference between the resulting condensation temperature (T_{COND}) and the current temperature in the T[N] array corresponding to the predetermined distillation profile 105 is compared 665 to the corresponding tolerance value (TOL[i]). If the difference is not below the tolerance value, then the reflux ratio is updated 670 (e.g., by incrementation, an optimization algorithm, error reduction method, or some combination thereof) and the distillation experiment is performed 660 again.

Once the difference between the condensation temperature and the corresponding temperature in the predetermined distillation profile 105 is below the corresponding tolerance value, then the current reflux ratio has been successfully determined, and the counter variable is incremented 675. The process continues until a reflux ratio value has been determined for each temperature-quantity pair. Once the last pass through the loop has been completed 630, then the resulting reflux ratio profile 685 is stored 680 and the process 600 is complete.

FIG. 7 depicts an exemplary method of determining a heat transfer coefficient (K) as at least one calibrating parameter for a distillation model based on a predetermined distillation profile and a composition profile of a base fluid. The method 700 begins by initializing 702 corresponding arrays of temperature (T[N]) and quantity (V[N], e.g., volume fraction, mole fraction, mass fraction) from the base distillation profile (DP_{BASE-P}) 105, where N is the length of the arrays corresponding to the number of temperature-quantity data pairs used to represent the base distillation profile 105. A counter variable (i) is initialized 704 (e.g., to zero, as depicted). A heat transfer coefficient (K) is initialized 706. In various embodiments the initial value of the heat transfer coefficient may be determined from a look up table, from an initial ‘guess’ algorithm, from a manual input, from published correlations of heat transfer coefficients of various compositions, or some combination thereof. In various embodiments one or more coefficients (e.g., K) may correspond to (physical) attributes additional to or other than heat transfer. A tolerance value (TOL) is initialized 708 with a predetermined tolerance threshold. The tolerance threshold may, for example, be input manually, be calculated according to predetermined rules, be retrieved from one or more data stores, or some combination thereof. A current error value (E_{C}) is initialized 710 to the tolerance threshold and a past error value (E_{P}) is initialized to zero. An experimental temperature array (T_{E}[N]) of length N, corresponding to temperatures which will be determined as outputs of the distillation model 125, is initialized 712. The experimental temperature array may, for example, be initialized to an ambient temperature, to zero, to another appropriate temperature, or some combination thereof.

A base fluid stream corresponding to the base composition profile 120 for the process simulation is initialized 714 to predetermined conditions (e.g., temperature, relative quantities of components, vapor fractions). The base composition profile 120 may, for example, be determined by method 300 as described in relation to FIG. 3, including, for example, using speciation method(s) described in relation thereto. An ambient temperature (T_{AMB}) is set 716 to a predetermined ambient temperature. The predetermined ambient temperature may, for example, correspond to a default ambient temperature, a standard-defined ambient temperature, a known ambient temperature for a specific distillation experiment, a manually input ambient temperature, and ambient temperature optimized for one or more specific distillation experiments, or some combination thereof.

A heat transfer coefficient determination loop (steps 718-740) is repeated until a difference between the current error (E_{C}) and the past error (E_{P}) is below 718 the tolerance threshold (TOL). When the difference between the errors is not below 718 the tolerance threshold, then the method checks 720 to determine if the current pass is the first pass through the loop, corresponding to the initial value of the past error (E_{P}=0). If the current pass is not the first pass through the loop 720, then the heat transfer coefficient (K) is updated. As depicted, the heat transfer coefficient is updated according to an optimization algorithm. For example, the optimization algorithm may adjust the heat transfer coefficient based on the difference calculated in step 718. In various embodiments an optimization algorithm may, for example, advantageously increase a speed of convergence of the distillation model 125 with the predetermined distillation profile 105.

Once the heat transfer coefficient has been updated 722, or if the current pass is the first pass through the loop 720, then a temperature calculation subloop (steps 724-732) is entered. The temperature calculation subloop generates an experimental temperature array (T_{E}) corresponding to a tested distillation profile for a given K. A fraction condensed (F_{COND}) is calculated 724. As depicted, the fraction condensed corresponds linearly to a difference between a current condensation temperature (T_{COND}) in the distillation model 125 (e.g., a temperature in a distillation column, such as in the neck of a distillation flask) and the ambient temperature (T_{AMB}). The fraction condensed may, for example, correspond to a quantity of the fluid in the distillation experiment which remains as vapor, condenses back into a liquid reservoir, or some combination thereof. In various embodiments, the distillation model 125 a distillation column a distillation experiment may be represented by one or more condenser units. The condenser unit(s) may, for example, be correlated to a fraction condensed. For example, fraction condensed may correspond to a condenser duty. The condenser duty may, for example, be dynamically solved in a condenser process simulation module (e.g., such as in the ProMax® software package commercially available from Bryan Research and Engineering, LLC of Bryan, Tex.).

The distillation experiment represented by the distillation model 125 is performed 726. The counter variable (i) is incremented 728 and the process checks 730 to determine if the entire experimental temperature array has been generated. If the entire experimental temperature array has not been generated, the inlet stream of the distillation model 125 is set 731 to a residual from the previous temperature and the current condensation temperature (T_{COND}) is stored 732 in the corresponding location in the temperature array (T_{E}[i]). Once the entire temperature array (T_{E}) has been generated 730 for the current K, then the counter variable is reinitialized 734, the past error is updated to the current error value 736, and the current error is reinitialized 738.

The method 700 then enters an error calculation subloop (steps 740-744) which calculates a current error value (E_{C}) for the present K. If the error has not been determined for all N temperature points 740, then the current error is updated 742 by accumulating the square of the difference between the temperature of the predetermined distillation profile and the experimental temperature for the current point. the counter variable is an incremented 744. Once the error has been accumulated 742 for all points in the array 740, the counter variable is reinitialized 746 and the difference between the current error and the past error is compared to the tolerance threshold 718. The loop (718-746) repeats until K is calibrated such that the distillation model 125 generates a test distillation profile which corresponds to the predetermined distillation profile 105 within the tolerance threshold.

Once the tolerance threshold has been reached 718, then the calibrated K value is stored 748 as a calibration parameter 750 appropriate for the base composition profile 120 and the predetermined distillation profile 105. Accordingly, various embodiments may advantageously determine a heat transfer coefficient (K) which calibrates a distillation model 125 to a predetermined distillation profile 105.

In various embodiments the heat transfer coefficient may be determined (e.g., steps 718-746), for example, by calculating a heat transfer coefficient such that the quantity of the distillation model 125 at the current step matches a corresponding quantity at a corresponding temperature in the predetermined distillation profile 105, the temperature of the distillation model 125 at a current step matches a corresponding temperature to corresponding quantity in the predetermined distillation profiles 105, or some combination thereof. In various embodiments the heat transfer coefficient may be calculated using a steady state distillation modeling process (e.g., incrementing through temperature and varying the heat transfer coefficient until the quantity matches the predetermined distillation profile at the point corresponding to the current temperature, or vice versa varying quantity and matching temperature), a dynamic distillation modeling process (e.g., incrementing through time and varying the heat transfer coefficient until temperature and quantity correspond to a point in the predetermined distillation profile 105), or some combination thereof.

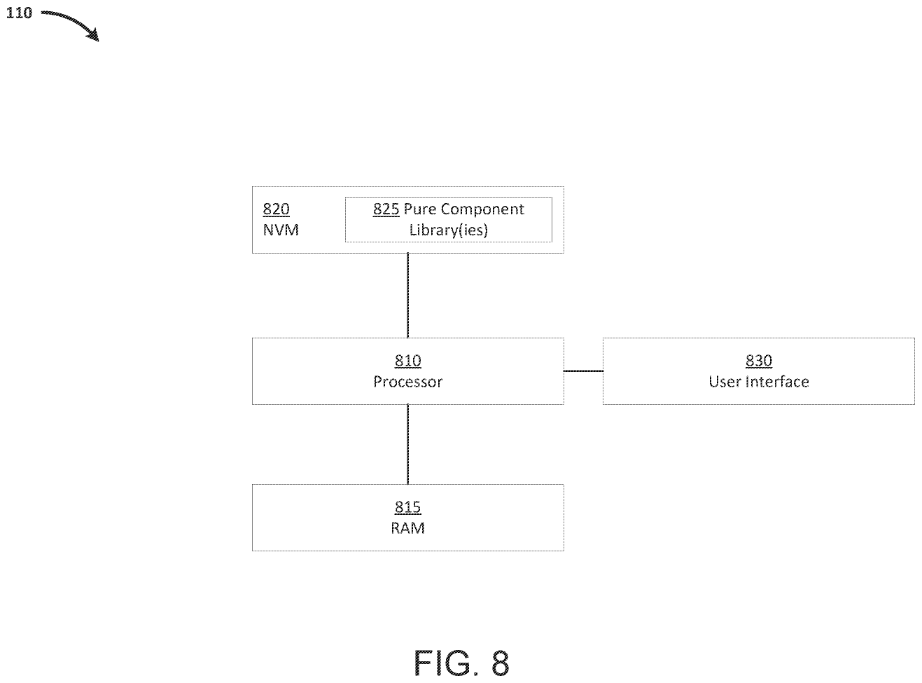

FIG. 8 depicts an exemplary hardware block diagram of an automatic laboratory module 110. In the depicted example, the automatic laboratory module 110 includes processor 810, which is operably connected to a memory module 815 and a storage module 820. In various embodiments the processor 810 may include one or more interconnected and/or separate physical processor units. As depicted, the memory module 815 includes random access memory (RAM) and the storage module 820 includes non-volatile memory.

As depicted, storage module 820 includes a pure component library or libraries 825. For example, storage module 820 may contain one or more databases including candidate pure components and associated characteristics. The processor 810 is operably connected to and configured to interact with user interface 830. The user interface 830 may, for example, allow a user to input information, to view results, or some combination thereof. In various embodiments the processor 810 is configured to generate a graphical representation at least of the mixture distillation profile 140. The automatic lab module 110 may, for example, be provided with at least one program of instructions (e.g., stored in memory 820) that, when executed by the processor 810, cause operations to be performed to execute methods which may include, by way of example and not limitation, methods described in relation to FIGS. 2-7.

Although various embodiments have been described with reference to the figures, other embodiments are possible. Although an exemplary system 100 has been described with reference to FIG. 1, other implementations may be deployed in other industrial, scientific, medical, commercial, and/or residential applications. Although an example of a system, which may be portable, has been described with reference to the above figures, other implementations may be deployed in other processing applications, such as desktop and networked environments.

In various embodiments a composition profile may include a plurality of data objects corresponding to associated molecular compounds. A composition profile may also include, by way of example and not limitation, one or more chemical attributes (e.g., flash point, boiling point, molecular structure, interaction parameter(s)/activity coefficient). In various embodiments a single composition profile may include, by way of example and not limitation, about 1, 5, 10, 15, 20, 25, 30, 40, 50, 100, or more unique pure components. In some embodiments a library of pure components from which a composition profile may be constructed may include, by way of example and not limitation, about 5, 10, 15, 20, 25, 30, 40, 50, 100, 250, 500, 1000, or more unique pure components. In various embodiments a candidate pure component may be selected to represent a composition in a composition profile at least partially based on the presence of a non-zero interaction parameter with at least one additive of interest. In various embodiments a base composition profile may be modified when generating a corresponding mixture composition profile to account for differences between heat capacities of the base fluid (e.g., gasoline) and additive(s) (e.g., ethanol) and/or latent heat of vaporization.

In various embodiments a distillation profile may include a plurality of matched temperature-quantity points. Quantity may, by way of example and not limitation, include mass fraction, volume fraction, mole fraction, or some combination thereof. In various embodiments a distillation profile may, for example, be a plurality of temperature-quantity points derived from a continuous curve representing a distillation relationship. In some embodiments, a distillation profile may relate energy to quantity. For example, energy may not be correlated to temperature.

In various embodiments the base fluid may include, by way of example and not limitation, a fuel, a hydrocarbon mixture, tallow, biofuel, other at least partially distillable fluids, or some combination thereof. In various embodiments the additive(s) may include, by way of example and not limitation, ethanol, propanol, methanol, methyl-2-butyl ether (MTBE), alcohol, other desired additives, or some combination thereof. In some embodiments the base fluid(s) and/or the additive(s) may only be in a fluid state in certain conditions.

Various embodiments may implement one or more of the methods described with reference to the figures herein to automatically adjust composition of a mixture by controlling, by way of example and not limitation, proportions of one or more additives (e.g., 135), one or more base fluids (e.g., 120), or some combination thereof, in a chemical process (e.g., representing an industrial chemical facility and/or system). such embodiments may, for example, advantageously maintain desired attributes corresponding to a target distillation curve of a resulting mixture based on changes composition of one or more input streams.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device, such as a CRT (cathode ray tube) or LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A computer program product comprising:
a program of instructions tangibly embodied on a non-transitory computer readable medium wherein, when the instructions are executed on a processor, the processor causes operations to be performed to automatically characterize distillation attributes of a mixture of a multicomponent hydrocarbon base fluid with at least one additive comprising ethanol, the operations comprising:
obtain a predetermined distillation profile corresponding to a predetermined fuel distillation protocol performed on the base fluid;
generate a first composition profile that defines pure chemical components representing the base fluid, comprising:
determine, according to predetermined speciation rules, a plurality of pure chemical components having predetermined thermodynamic interaction parameters with the at least one additive, and,
determine a relative amount of each of the plurality of pure chemical components;
generate a distillation model corresponding to the predetermined fuel distillation protocol and based on the first composition profile and predetermined distillation profile by determining at least one calibrating parameter of the distillation model such that the distillation model is calibrated to the predetermined distillation profile to generate a first distillation profile of the base fluid as a function of the first composition profile and the calibrating parameter; and,
apply the calibrated distillation model to a second composition profile of pure chemical components representing a mixture of the base fluid and the at least one additive to generate a second distillation profile as a function of the at least one calibrating parameter and the second composition profile.

2. The computer program product of claim 1, wherein the at least one calibrating parameter of the distillation model is a reflux ratio profile determined from the predetermined distillation profile such that a plurality of temperature-quantity points in the first distillation profile are within a predetermined threshold of corresponding temperature-quantity points in the predetermined distillation profile.

3. The computer program product of claim 1, wherein the at least one parameter of the distillation model is a heat transfer coefficient determined from the predetermined distillation profile such that a plurality of temperature-quantity points in the first distillation profile are within a predetermined threshold of corresponding temperature-quantity points in the predetermined distillation profile.

4. A computer program product comprising:
a program of instructions tangibly embodied on a non-transitory computer readable medium wherein, when the instructions are executed on a processor, the processor causes operations to be performed to automatically characterize distillation attributes of a mixture of a multicomponent base fluid with at least one additive, the operations comprising:
obtain a predetermined distillation profile of the base fluid;
obtain a first composition profile that defines pure chemical components representing the base fluid;
generate a distillation model based on the first composition profile and predetermined distillation profile by determining at least one calibrating parameter of the distillation model such that the distillation model is calibrated to the predetermined distillation profile to generate a first distillation profile of the base fluid as a function of the first composition profile and the calibrating parameter; and,
apply the calibrated distillation model to a second composition profile of pure chemical components representing a mixture of the base fluid and the at least one additive to generate a second distillation profile as a function of the at least one calibrating parameter and the second composition profile.

5. The computer program product of claim 4, wherein the at least one calibrating parameter of the distillation model is a reflux ratio profile determined from the predetermined distillation profile such that a plurality of temperature-quantity points in the first distillation profile are within a predetermined threshold of corresponding temperature-quantity points in the predetermined distillation profile.

6. The computer program product of claim 5, wherein the reflux ratio profile comprises a plurality of reflux ratios and corresponding quantity metric values.

7. The computer program product of claim 4, wherein the at least one parameter of the distillation model is a heat transfer coefficient determined from the predetermined distillation profile such that a plurality of temperature-quantity points in the first distillation profile are within a predetermined threshold of corresponding temperature-quantity points in the predetermined distillation profile.

8. The computer program product of claim 4, the operations further comprising:
generate the first composition profile, comprising:
determine, according to predetermined speciation rules, a plurality of pure chemical components having predetermined thermodynamic interaction parameters with the at least one additive; and,
determine a relative amount of each of the plurality of pure chemical components.

9. The computer program product of claim 8, wherein the thermodynamic interaction parameters comprise binary interaction parameters.

10. The computer program product of claim 8, wherein the thermodynamic interaction parameters correspond to parameters of an activity coefficient model.

11. The computer program product of claim 4, wherein generate a second distillation profile comprises, for a plurality of temperatures corresponding to the predetermined distillation profile, determining corresponding quantities of the mixture in a batch distillation process represented by the distillation model.

12. The computer program product of claim 4, wherein generate a second distillation profile comprises, for a range of time in a distillation process represented by the distillation model, determining corresponding quantity and temperature relationships of the mixture in the distillation process.

13. The computer program product of claim 4, wherein:
the predetermined distillation profile corresponds to results of at least one experiment performed according to the ASTM D86 standard, and,
the distillation model is configured to effect an electronically performed ASTM D86 experiment.

14. The computer program product of claim 13, wherein the first distillation profile and the second distillation profile represent results of the ASTM D86 experiment when electronically performed on the first composition profile and the second composition profile, respectively.

15. The computer program product of claim 4, wherein the first composition profile comprises hydrocarbon molecules.

16. The computer program product of claim 4, wherein the second composition profile comprises alcohol molecules.

17. A computer-implemented method configured to automatically characterize distillation attributes of a mixture of a multicomponent base fluid with at least one additive, the method comprising:
obtain a predetermined distillation profile of the base fluid;
obtain a first composition profile that defines pure chemical components representing the base fluid;
generate a distillation model based on the first composition profile and predetermined distillation profile by determining at least one calibrating parameter of the distillation model such that the distillation model is calibrated to the predetermined distillation profile to generate a first distillation profile of the base fluid as a function of the first composition profile and the calibrating parameter; and,
apply the calibrated distillation model to a second composition profile of pure chemical components representing a mixture of the base fluid and the at least one additive to generate a second distillation profile as a function of the at least one calibrating parameter and the second composition profile.

18. The computer-implemented method of claim 17, wherein the at least one calibrating parameter of the distillation model is a reflux ratio profile determined from the predetermined distillation profile such that a plurality of temperature-quantity points in the first distillation profile are within a predetermined threshold of corresponding temperature-quantity points in the predetermined distillation profile.

19. The computer-implemented method of claim 17, wherein the at least one parameter of the distillation model is a heat transfer coefficient determined from the predetermined distillation profile such that a plurality of temperature-quantity points in the first distillation profile are within a predetermined threshold of corresponding temperature-quantity points in the predetermined distillation profile.

20. The computer program product of claim 17, the method further comprising:
generate the first composition profile, comprising:
determine, according to predetermined speciation rules, a plurality of pure chemical components having predetermined thermodynamic interaction parameters with the at least one additive; and,
determine a relative amount of each of the plurality of pure chemical components.
